(19) United States
(12) Patent Application Publication
(10) Pub. No.: US 2009/0264909 A1
(43) Pub. Date: Oct. 22, 2009

(54) Ultrasonic shears stop pad

(76) Inventor: Jean Michael Beaupre, Alexandria, KY (US)

Correspondence Address: Jean M. Beaupre, Suite 412, 4480 Lake Forest Dr., Cincinnati, OH, 45242, US

(21) Appl. No.: 12/386,468

(22) Filed: Apr. 17, 2009

Related U.S. Application Data
(60) Provisional application No. 61/124,643, filed on Apr. 18, 2008.

(51) Int. Cl. A61B 17/3201

(52) U.S. Cl. 606/169

(57) ABSTRACT

An ultrasonic-surgical-shears stop pad has a stop-pad body including a base material and at least one filler material. An alternate ultrasonic-surgical-shears stop pad has a stop-pad body adjacent to tissue pad. An ultrasonic surgical shears includes an ultrasonic surgical blade and a clamping arm which is operable to open and close toward the blade and which has a transversely and resiliently flexible distal tip. An alternate ultrasonic surgical shears includes an ultrasonic surgical blade, a clamping arm operable to open and close toward the blade, and a stop pad attached to the clamping arm and having a blade contact surface, wherein at least a portion of the stop pad is resiliently flexible in a direction substantially perpendicular to the blade contact surface.

This application hereby claims the priority of U.S. Provisional Application 61/124,643 filed on Apr. 18, 2008. U.S. Provisional Application 61/124,643 is incorporated by reference.

### FIELD OF THE INVENTION

The present invention relates, in general, to ultrasonic devices and, more particularly, to methods and devices that provide ultrasonic shears with improved cutting and hemostasis.

### BACKGROUND OF THE INVENTION

Ultrasonic surgical instruments are known which include an ultrasonic surgical shears having an ultrasonic surgical blade, a clamping arm operable to open and close toward the blade, and a polytetrafluoroethylene tissue pad which is attached to the clamping arm and which includes a clamping surface. The clamping arm exerts a clamping force on a blood vessel which is positioned between the clamping surface of the tissue pad and the blade. The result of the ultrasonically-vibrating ultrasonic surgical blade and the clamping force on the blood vessel is a coaptation of the blood vessel (a bringing together of the walls of the blood vessel), a transection (a cutting) of the coapted blood vessel, and a coagulation (a sealing) of the coapted cut ends of the blood vessel. At the completion of a tissue transection, the ultrasonically-vibrating ultrasonic surgical blade contacts and cuts away some of the polytetrafluoroethylene tissue pad because of the frictional abrasion and frictional heat generated by the blade vibrating against the tissue pad. Exemplary devices are described in U.S. Pat. Nos. 5,322,055 and 6,325,811, the contents of which are incorporated herein by reference.

Devices to address frictional abrasion and frictional heat in the tissue pad have been described in U.S. Patent Publication 20050192610. However, this method has the limitation of requiring the pad material to be optimized for tissue contact and for frictional effects.

Still, scientists and engineers continue to seek improved ultrasonic surgical shears and improved clamp arms for ultrasonic surgical shears.

### SUMMARY OF THE INVENTION

The present invention comprises a stop pad connected to clamping arm and separate from the tissue pad. Said stop pad contacts the ultrasonic surgical blade but does not engage tissue and may be optimized through material selection, geometry, and position to reduce negative frictional effects independent of tissue pad.

A first embodiment of an ultrasonic-surgical-shears stop pad of the invention includes an ultrasonic-surgical-shears stop pad body proximal to the tissue pad. Said stop pad having a base material with either no filler or at least one filler material which is a different material from the base material. The stop pad base material may be the same or different than the tissue pad material

A second embodiment of an ultrasonic-surgical-shears stop pad of the invention includes an ultrasonic-surgical-shears stop pad body offset from the tissue pad with respect to the blade. Said stop pad having a base material with either no filler or at least one filler material which is a different material from the base material. The stop pad base material may be the same or different than the tissue pad material

A third embodiment of an ultrasonic-surgical-shears stop pad of the invention includes an ultrasonic-surgical-shears with tissue pad shaped such that tissue is prevented from touching the stop pad.

Several benefits and advantages are obtained from one or more of the embodiments of the invention. Having a stop pad not in contact with tissue allows material to be chosen with a different hardness, stiffness, lubricity, dynamic coefficient of friction, heat transfer coefficient, abradability, heat deflection temperature, and/or melt temperature to improve the wearability of the tissue pad which is important when high clamping forces are employed because tissue pads wear faster at higher clamping forces than at lower clamping forces. Furthermore, the stop pad surface need not be parallel or conformal to the blade surface at the point of contact. Applicants found that by through proper material selection, geometry, and position of the stop pad, tissue pad wearability is improved. Furthermore, vessel coaptation and transaction can be optimized by proper material selection, geometry, and position of the stop pad independent of the material selection, geometry, and position of the tissue pad.

The present invention has, without limitation, application in straight or curved ultrasonic surgical blades as disclosed in the patents incorporated by reference and further in hand-activated instruments as well as in robotic-assisted instruments.

### BRIEF DESCRIPTION OF THE DRAWINGS

The features of the invention may be set forth with particularity in the appended claims. The invention itself, however, both as to organization and methods of operation, together with further objects and advantages thereof, may best be understood by reference to the following description, taken in conjunction with the accompanying drawings in which:

FIG. 1 is a perspective view of an Ultrasonic shears and transducer;

FIG. 2 is a partial isometric view of the invention;

FIG. 3 is a partial section view of the invention;

FIG. 4 is a magnified partial section view of the invention;

FIGS. 5A-I are magnified partial section views of various embodiments of the invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is understood that any one or more of the following-described embodiments, examples, etc. can be combined with any one or more of the other following-described embodiments, examples, etc.

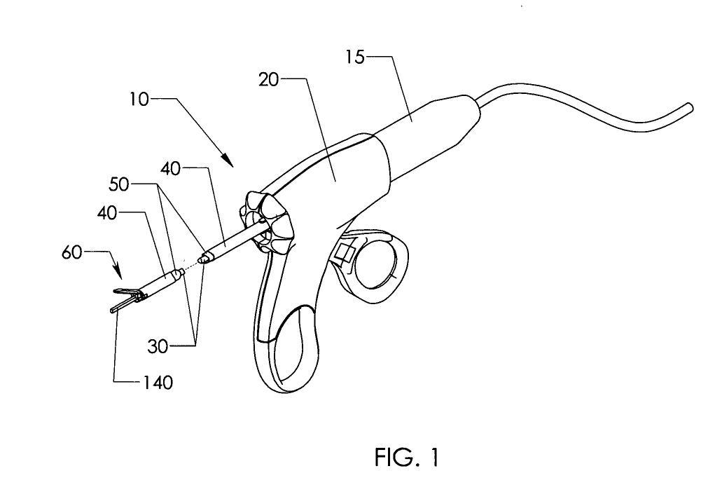

Referring now to the Figures, in which like numerals indicate like elements, FIG. 1 illustrates an ultrasonic shears 10 and transducer 15. Ultrasonic shears 10 include a handle 20, waveguide 30, first member 40, second member 50, and shears mechanism 60. Transducer 20 produces ultrasonic energy, which is transmitted by waveguide 30 to blade 140. First member 40 and second member 50 act together to actuate shears mechanism 60 to open and close.

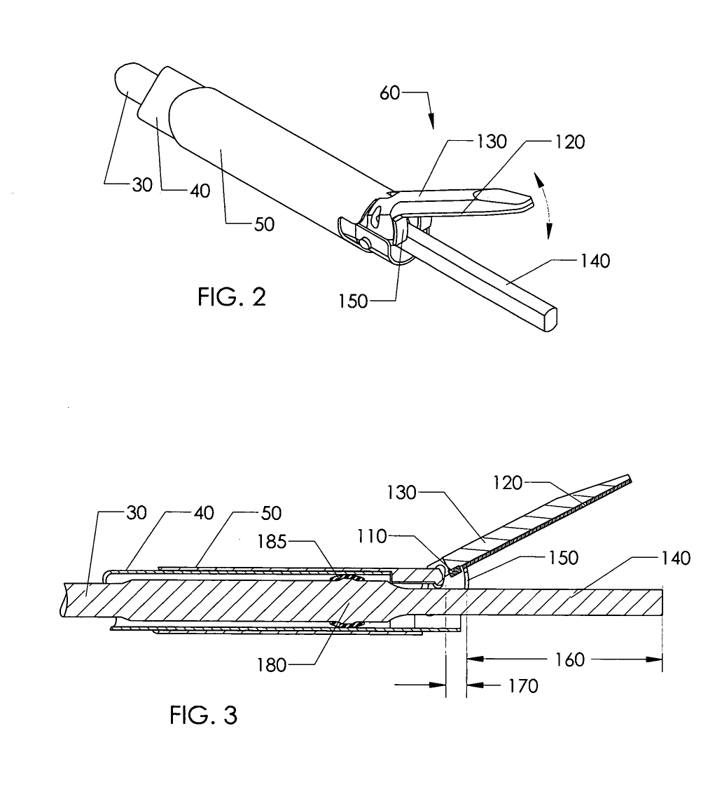

FIG. 2. and FIG. 3 illustrate a first embodiment of an ultrasonic-surgical-shears stop pad 110 of the invention. The ultrasonic-surgical-shears tissue pad 120 is interposed between the clamp arm 130 and the blade 140. Stop pad 130 is interposed between the clamp arm 130 and the blade 140 and is spaced from tissue pad 120. Clamp arm 130, stop pad 110, and tissue pad 120 are operable to open and close toward the blade 140. Space between tissue pad 120 and blade 140 adapted to grasp, manipulate, and treat tissue defines the therapeutic region 160. A proximal portion tissue pad 120 may occlude blade 140 to define tissue pad deflection 150. Therapeutic region 160 may be bounded by tissue pad deflection 150. Space between stop pad 110 and blade 140 define a non-therapeutic region 170. Portions of blade 140 having relatively little motion define node 180. Node 180 may be supported by first member 40 or second member 50 or surrounded by support ring 185 which is in turn supported by first member 40 or second member 50.

In one example of the embodiment of the ultrasonic-surgical-shears stop pad 110 of FIG. 2 and FIG. 3, stop pad 110 will contact blade 140 when clamp arm 130 is actuated closed. Blade 140 surface motion correlates to distance from node 180, with motion generally decreasing with proximity to node 180. By placing stop pad 110 proximal to tissue pad 120, stop pad 110 will experience less blade 140 motion than tissue pad 120.

In one illustration of the ultrasonic-surgical-shears tissue pad 110 of FIG. 2 and FIG. 3, the base material consists essentially of a thermoset plastic material. Alternative base materials include PTFE, ultra high molecular weight polyethylene polyimide material or any material exhibiting a sufficient combination of hardness, stiffness, lubricity, dynamic coefficient of friction, heat transfer coefficient, abradability, heat deflection temperature, and/or melt temperature. Homogeneous and non-homogeneous mixtures with sufficient combination of hardness, stiffness, lubricity, dynamic coefficient of friction, heat transfer coefficient, abradability, heat deflection temperature, and/or melt temperature are also acceptable.

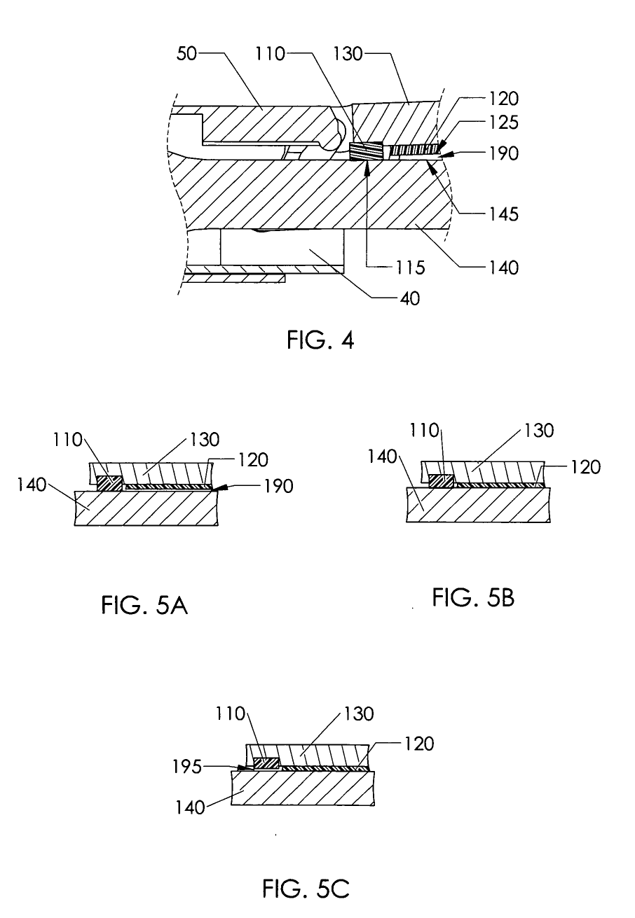

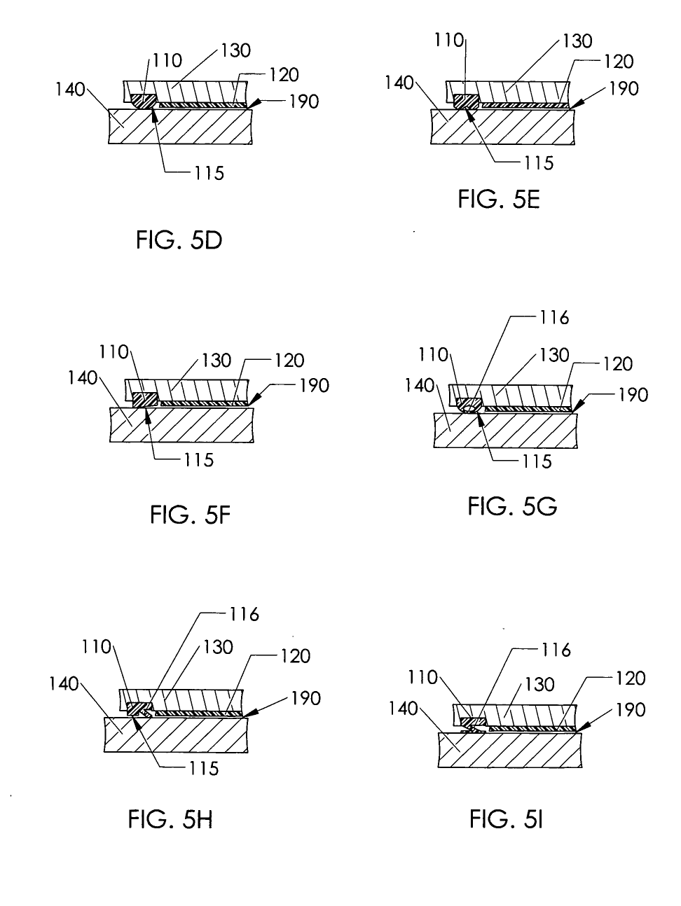

In one example of the embodiment of the ultrasonic-surgical-shears stop pad 110 of FIG. 4 and FIG. 5A, stop pad 110 may be configured through proper selection of geometry and position such that stop pad surface 115 contacts blade surface 145, creating gap 190 between blade surface 145 and tissue pad surface 125. As clamp arm 130 is actuated towards blade stop pad 110 may contact blade 140 prior to tissue pad 120 contacting the blade 140, allowing maximum force to be applied to tissue until it is compressed to a particular thickness. Force is then limited by the compliance of stop pad 110 and blade 140.

In one example of the embodiment of the ultrasonic-surgical-shears stop pad 110 of FIG. 5B, stop pad 110 may be configured through proper selection of geometry and position such that as clamp arm 130 is actuated towards blade Stop pad 110 may contact blade 140 simultaneous to tissue pad 120 contacting the blade 140, distributing force across tissue pad 120 and stop pad 110.

In one example of the embodiment of the ultrasonic-surgical-shears stop pad 110 of FIG. 5C, stop pad 110 may be configured through proper selection of geometry and position such that as clamp arm 130 is actuated towards blade Stop pad 110 may contact blade 140 subsequent to tissue pad 120 contacting the blade 140, creating gap 195, allowing maximum force to be applied to tissue at any thickness. Compliance of tissue pad 120 and blade 140 allows stop pad 110 to contact blade 140 at a maximum force. Additional force is then distributed across tissue pad 120 and stop pad 110.

In further examples of the embodiment of the ultrasonic-surgical-shears stop pad 110 of FIG. 5D through FIG. 5H, stop pad 110 may be configured through proper selection of geometry and position such that stop pad surface 115 is not conformal to blade surface 145.

In further examples of the embodiment of the ultrasonic-surgical-shears stop pad 110 of FIG. 5G through FIG. 5I, stop pad 110 may be configured through proper selection of geometry and position to include deformable structure 116. Deformable structure 116 may be composed of cantilever beams, voids, springs, elastomeric composites, or similar structures or combinations thereof.

Several benefits and advantages are obtained from one or more of the embodiments of the invention. Having a stop pad distinct from a tissue pad allows the stop pad base material and the at-least-one filler material to be chosen with a different hardness, stiffness, lubricity, dynamic coefficient of friction, heat transfer coefficient, abradability, heat deflection temperature, and/or melt temperature independent of tissue pad requirements to improve the wearability of the tissue pad which is important when high clamping forces are employed because tissue pads wear faster at higher clamping forces than at lower clamping forces. Furthermore, the stop pad surface need not be parallel or conformal to the blade surface at the point of contact. Applicants found that by through proper material selection, geometry, and position of the stop pad, tissue pad wearability is improved. Furthermore, vessel coaptation and transaction can be optimized by proper material selection, geometry, and position of the stop pad independent of the material selection, geometry, and position of the tissue pad. Having a flexible clamping arm and/or a flexible tissue pad should also improve the wearability of the tissue pad due to the ability of the flexible member to more evenly distribute the load across the entire surface of the tissue pad.

While the present invention has been illustrated by a description of several embodiments, it is not the intention of the applicants to restrict or limit the spirit and scope of the appended claims to such detail. Numerous other variations, changes, and substitutions will occur to those skilled in the art without departing from the scope of the invention. For instance, the ultrasonic surgical shears and the stop pad of the invention have application in robotic assisted surgery taking into account the obvious modifications of such systems, components and methods to be compatible with such a robotic system. It will be understood that the foregoing description is provided by way of example, and that other modifications may occur to those skilled in the art without departing from the scope and spirit of the appended Claims.

What is claimed is:

1. An ultrasonic-surgical-shears stop pad comprising: an ultrasonic-surgical-shears stop pad adjacent to tissue pad.

2. The ultrasonic-surgical-shears stop pad of claim 1, wherein the at-least-one filler material has at least one property which has a different value from that of the at-least-one property of the base material, and wherein the at-least-one property is chosen from the group consisting of: hardness, stiffness, lubricity, dynamic coefficient of friction, heat transfer coefficient, abradability, heat deflection temperature, and melt temperature.

3. The ultrasonic-surgical-shears stop pad of claim 1, wherein the base material has a heat deflection temperature greater than 500 degrees Fahrenheit.

4. The ultrasonic-surgical-shears stop pad of claim 1, wherein stop pad does not contact the blade simultaneous with tissue pad

5. The ultrasonic-surgical-shears stop pad of claim 1, wherein the surface of said stop pad contacting blade is not conformal to said blade

6. The ultrasonic-surgical-shears stop pad of claim 1, wherein the base material consists essentially of a thermoset plastic material.

7. The ultrasonic-surgical-shears stop pad of claim 6, wherein the base material consists essentially of a polyimide material.

8. An ultrasonic-surgical-shears stop pad and tissue pad comprising: an ultrasonic-surgical-shears stop pad body includes a first material and wherein the tissue pad includes a second material which is a different material from the first material.

9. The ultrasonic-surgical-shears stop pad of claim 8, wherein the first region consists essentially of the first material and wherein the second region consists essentially of the second material.

10. The ultrasonic-surgical-shears stop pad of claim 8, wherein the first material has at least one property which has a different value from that of the second material, and wherein the at-least-one property is chosen from the group consisting of: hardness, stiffness, lubricity, dynamic coefficient of friction, heat transfer coefficient, abradability, heat deflection temperature, and melt temperature.

11. The ultrasonic-surgical-shears stop pad of claim 8, wherein the first material has a heat deflection temperature greater than 500 degrees Fahrenheit.

12. The ultrasonic-surgical-shears stop pad of claim 8, wherein wherein the surface of said stop pad contacting blade is not conformal to said blade

13. The ultrasonic-surgical-shears stop pad of claim 8, wherein stop pad does not contact the blade simultaneous with tissue pad

14. The ultrasonic-surgical-shears stop pad of claim 8, wherein the first material consists essentially of a thermoset plastic material.

15. The ultrasonic-surgical-shears stop pad of claim 14, wherein the base material consists essentially of a polyimide material.

16. The ultrasonic-surgical-shears stop pad of claim 8, wherein the stop pad may flex

17. An ultrasonic surgical shears comprising: a) an ultrasonic surgical blade; b) a clamping arm operable to open and close toward the blade; and c) a stop pad attached to the clamping arm and having a clamping surface, wherein at least a portion of the stop pad is resiliently flexible in a direction substantially perpendicular to the clamping surface.

18. The ultrasonic surgical shears of claim 17, wherein the stop pad comprises a stop pad body including a base material and at least one filler material, wherein the at-least-one filler material has at least one property which has a different value from that of the at-least-one property of the base material, and wherein the at-least-one property is chosen from the group consisting of: hardness, stiffness, lubricity, dynamic coefficient of friction, heat transfer coefficient, abradability, heat deflection temperature, and melt temperature.

19. The ultrasonic surgical shears of claim 17, wherein the clamping arm comprises a tissue pad body and an adjacent stop pad, wherein the tissue pad is adapted to treat tissue and the stop pad is not adapted to treat tissue.
